(19) United States
(12) Patent Application Publication
(10) Pub. No.: US 2008/0131496 A1
(43) Pub. Date: Jun. 5, 2008

(54) TOPICAL APPLICATION OF MELATONIN DIRECTLY OR IN LIPOSOMES FOR THE AMELIORATION OF ITCHING AND HISTAMINE AND NON HISTAMINE RELATED INFLAMMATORY SKIN CHANGES

(76) Inventor: F. Timothy Guilford, Palo, CA (US)

Correspondence Address: BROOKE SCHUMM III;Daneker, McIntire, Schumm, Prince, Goldstein et al, ONE NORTH CHARLES STREET, SUITE 2450, BALTIMORE, MD, 21201, US

(21) Appl. No.: 11/860,521

(22) Filed: Sep. 24, 2007

Related U.S. Application Data
(60) Provisional application No. 60/826,725, filed on Sep. 22, 2006.
(60) Provisional application No. 60/867,414, filed on Nov. 28, 2006.
(60) Provisional application No. 60/974,756, filed on Sep. 24, 2007.

(51) Int. Cl. A61K 9/127; A61K 31/404; A61P 17/00

(52) U.S. Cl. 424/450; 514/419

(57) ABSTRACT

The invention is a topical therapeutically effective amount of melatonin encapsulated in a liposome applied topically to an area of skin affected by immunologic response, radiation treatment induced dermatitis, acne, insect bite or other irritant stimulus such as sunburn in order to reduce itching. The invention also proposes a topical therapeutically effective amount of melatonin and reduced glutathione encapsulated in a liposome applied topically to an area of skin for relief of those afflictions.

### CONTINUATION DATA

This application claims benefit of applications of the same name filed by the inventor as provisional applications in the U.S. of America as Nos. 60/826,725 filed on Sep. 22, 2007 and 60/867,414 filed on Nov. 28, 2006, and 60/974,756 filed on Sep. 24, 2007. For U.S. purposes and any other country where permitted, the application is a continuation-in-part of those applications.

### SUMMARY OF INVENTION

The invention is the use of a topical therapeutically effective amount of melatonin encapsulated in a liposome applied topically to an area of skin affected by acne, insect bite or other irritant stimulus such as sunburn in order to reduce itching.

### TECHNICAL FIELD

The invention relates to the field of delivery of a nutrient substance, melatonin, either directly or preferably encapsulated in a liposome for the amelioration of itching and similar irritant responses that occur after insect bite or sunburn. The delivery may be accomplished using an aqueous spray suspension of the liposome melatonin complex or a cream for topical application.

### BACKGROUND

Itch, also called pruritis, is defined as “an unpleasant cutaneous sensation which provokes the desire to scratch” (Rothman). The entire mechanism of itching, medically known as pruritis, remains elusive, but there is clearly a triggering mechanism related to histamine release in the skin (Twycross). While itching is associated with several disease states the most common form is related to insect bite or other similar irritant stimulation to the skin. This topical application can be used to relieve the itching associated with healthy, normal healing of wounds. This helps reduce the urge to scratch and disrupt normal healing.

Histamine is the main mediator for itch after insect bite reactions and in other forms of itching such as urticaria. This has been determined by observation of dose response to intracutaneous histamine injections (Simone, Handwerker) and relief of symptoms by antihistamines (Twycross).

Most insect stings are non-allergic manifestations of the venom's toxic effects that result in erythema, pain and swelling at the sting site (Ellis). At the same time, insect bites such as bee venom and fire ant bites are well known to elicit an immunologic response ranging from local reactions to anaphylaxis, all initiated by histamine release from mastocytes in the skin.

Histamine release in the skin is a common response to many noxious stimuli to the skin. For example, histamine is released from mast cells in the skin after the application of ultraviolet light and heat to the skin to approximate levels that would occur with sun burn (Glover). Thus, the itching and irritation that accompanies sunburn is also a histamine response, although the stimulus is due to a physical stimulation as opposed to an immunologic trigger.

The local regulation of immune function is related to release of histamine from the immune cells called mast cells or mastocytes. These cells are located in all tissues of the body and an increase in their presence in a disease state called mastocytosis can lead to excess release of histamine and severe allergic reactions such as anaphylaxis (Ellis).

Because the initiation of itching after insect sting is mediated primarily by histamine release, antihistamines have become the primary method of treating itching after stings. The administration of antihistamines can be done either systemically or topically at the site of itching.

The invention utilizes the application of melatonin as a new method of achieving control of itching in the skin associated with insect bites, sunburn and similar irritations. There are no prior reference to the use of topical application of liposomal melatonin for the purpose of ameliorating itching and the preferred method of delivery that is encapsulation of the melatonin allows for an efficient delivery of the invention.

### Background for Liposomes

A liposome is a microscopic fluid-filled pouch whose walls are made of one or more layers of phospholipid materials identical to the phospholipid that makes up cell membranes. Lipids can be used to deliver materials such as drugs to the body because of the enhanced absorption of the liposome. The outer wall of the liposome is fat soluble, while the inside is water-soluble. This combination allows the liposome to become an excellent method for delivery of water-soluble materials that would otherwise not be absorbed into the body. A common material used in the formation of liposomes is phosphatidylcholine, the material found in lecithin. A more detailed description of the constituents of this invention is provided.

The observation that the topical application of liposome encapsulated melatonin would effective for itching is a surprise, as melatonin is most commonly associated with oral ingestion for systemic use. Although melatonin has been incorporated in some cosmetic formulations, as will be reviewed, its application in a spray lotion form that will ameliorate itching is not obvious. The spray liposomal melatonin was originally designed for oral use, and thus the concept that it might be effective for topical use has not been developed or claimed previously.

Melatonin is a hormone produced primarily by the pineal gland in the brain and was known initially for its relationship with sleep. Additional functions for melatonin have been identified in the last several years.

The bioactivity of melatonin is related to numerous behavioral, endocrinological, and immune processes. These activities can be mediated by receptor dependent or independent mechanisms. Melatonin is relatively small and has an amphiphilic nature that makes it very lipid soluble, so melatonin can easily pass through tissues and reach all of the cell compartments. While melatonin is well known for its sleep inducing qualities, it has also been shown to be a potent antioxidant.

The antioxidant activity of melatonin is mediated in several ways. Melatonin has direct anti-oxidant capacity by scavenging reactive oxygen species and nitrogen-based reactants. It has also been shown to induce additional antioxidants, such as superoxide dismutase, glutathione peroxidase, glutathione reductase and catalase. Melatonin has also been shown to induce the production of glutathione and is able to maintain a high reduced glutathione to oxidized glutathione ratio (GSH/GSSG) (Slominski)

Thus, the antioxidant capacity of melatonin has been shown to be quite complex. It is involved not only as a direct antioxidant, but by stimulating the formation of additional antioxidants, becomes involved in the control and modulation of processes which maintain the balance between antioxidant and pro-oxidant species. These actions contribute to melatonin's ability to maintain and restore antioxidant stability of cells and tissues under high oxidation stress (Allegra).

Studies of stressed skin using skin flaps placed on laboratory animals to create vascular compromise has found increased levels of oxidation stress markers such as malondialdehyde (MDA) and nitric oxide and lower levels of glutathione and glutathione peroxidase when compared to controls. In these studies, the administration of melatonin to the animals that had their pineal glands removed showed increased levels of glutathione and decreased levels of MDA and nitric acid than similar animals without the administration of melatonin (Gurlek).

Melatonin has been used systemically primarily for amelioration of sleep related problems. Animal studies have demonstrated that systemic melatonin has anti-inflammatory qualities (El-Shenawy). The study demonstrated that the effect of systemic melatonin after the injection of a noxious stimulant into the paw of the laboratory animal resulted in reduction of swelling and edema. The effect seemed to be similar to the anti-inflammatory effect of indomethacin, a non-steroidal anti-inflammatory drug (NSAID).

Prostaglandins (PGs) are a group of modified fatty acids that act as messengers involved in a number of responses including the inflammatory response to invading particles. The effect of prostaglandins is to dilate small blood vessels in the vicinity of an injury and causes the blood vessels to become leakier, allowing the infiltration of leukocytes. PGs are made with the help of a key enzyme called cyclooxygenase (COX) that catalyzes two sequential steps in the formation of PGs from arachidonic acid (AA). The enzyme exists in two forms, COX-1 which is localized to endothelial cells and platelets and an inducible form, known as COX-2. COX-2 is induced by a variety of stimuli in response to cell activation and inflammation.

COX-2 activity is joined by the inducible formation of nitric oxide synthase (iNOS) as important mediators of inflammation. iNOS creates nitric oxide from L-arginine. Over production of PGs and NP during inflammation leads to the production of fever, pain and edema (Kiefer). Methods of controlling COX-2 and iNOS are particularly useful for controlling the inflammatory process. A major mechanism of the action of NSAIDs is the inhibition of biosynthesis of PGs.

The more specific inhibition of COX-2 is desirable as opposed to the non specific inhibition of both COX-1 and COX-2 as the inhibition of COX-1 is associated with the side effects of damage to gastric mucosa, kidneys and platelet dysfunction.

It has been shown that melatonin inhibits the activation of the pro-inflammatory enzymes COX-2 and iNOS in macrophage cells after exposure to Lipopolysaccharides (LPS) toxin known to stimulate inflammation; at the same time melatonin does not inhibit COX-1 function (Mayo). A similar activity was noted with the melatonin metabolites, N1-acetyl-N2-formyl-5-methoxykynuramine (AFMK) and N1-acetyl-5-methoxykynuramine (AMK).

The toxin of poison oak, Rhus toxicodendron called urushiol is a phenolic lipid known to create an inflammatory reaction on contact with the skin. The oil apparently stimulates a delayed type hypersensitivity that is mediated by T cells, and thus requires previous exposure to trigger a response. Application of the liposomal melatonin invention during the early phases of the onset of the reaction ameliorates the allergic reaction and reduces the itching and inflammation due to poison oak. Case examples are later discussed and go into greater detail.

immunologically, melatonin receptors have been observed on the immune lymphocytes known as T-helper cells. Activation of the melatonin receptors results in release of T-helper cell Type 1 (TH1) cytokines, such as gamma-interferon (gamma-IFN) and IL-2, as well as of novel opioid cytokines (Maestroni). Melatonin also increases the release of IL-12 from monocytes, which is also associated with the TH1 response. However, the Maestroni article points out that it has not been clearly delineated if the effect of melatonin is purely enhancement of the TH1 function.

In contrast to melatonin, histamine is known to increase the response of the TH2 type of inflammatory reaction (Packard). Histamine increases the secretion of cytokines which are associated with an increase in T helper type 2 (TH2) cells. Histamine increases the release of IL-4 (interleukin-4), IL-5, IL-10 and IL-13 and inhibits the production of TH1 cytokines IL-2 and IFN-gamma (interferon-gamma) and monokine IL-12.

The research literature relates that there is still debate about the mechanism of action of melatonin in terms of its activity in stimulating TH1 response. However, the clinical observation of the effect of the topical application of this invention to insect bite sites of itching support the concept that there is a rapid resolution of both histamine effect and a down regulation of the immune mechanisms associated with histamine release, suggesting a switch to TH1 mechanisms and decreased inflammation.

### Background of Immune Function

The immune system contains a form of immunity called adaptive or acquired that refers to antigen-specific defense mechanisms that take several days to become protective and are designed to remove specific antigens. This is the immunity that one develops for life long protection. There are two major branches of the adaptive immune response: humoral and cell-mediated immunity.

Cell-mediated immunity involves the production of cytotoxic T-lymphocytes, activated macrophages, activated NK (Natural Killer) cells, and cytokines in response to an antigen and is mediated by T-lymphocytes. The cytokines released by the cells associated with this type of immune response are called TH1.

Humeral immunity involves the production of antibody molecules in response to an antigen, and is mediated by B-lymphocytes. This type of response is characterized by cells that release cytokines associated with the TH2 response.

TH1 lymphocytes, the cellular immune response cells, recognize antigens such as viruses presented by macrophages and activate and increase cell-mediated immunity by producing cytokines such as interleukin-2 (IL-2), interferon-γ, (IFN-γ), lymphotoxin and tumor necrosis factor-α and β. These cytokines enable T8-lymphocytes to differentiate into cytotoxic T-lymphocytes capable of destroying infected host cells, as well as activating cytotoxic T-lymphocytes and NK cells.

The cytokines released by TH2 lymphocytes include IL-2, 4, 5, 10, and 13 that promote antibody production. These cytokines enable and activate B-lymphocytes and result in the production of antigen specific antibodies.

The balance between these two subsets of lymphocytes plays a crucial role in how well the body defends against certain infections. For example, TH1 cells are needed to produce IFN-γ, which prompts the release of TNF (Tumor Necrosis Factor). TNF encourages the formation of toxic forms of oxygen, called reactive oxygen species (ROS) that are capable of destroying microorganisms such as viruses. Conversely, the cytokines released by the TH2 cells such as IL4 can actually slow the microbe killing activity related to IFN-γ.

Inflammatory states which persist for prolonged periods of time without resolving the triggering event and results in damage to cells and tissues are called chronic inflammation. The prolonged itching that is associated with many insect bites can be viewed as a form of chronic inflammation.

### Effect of Insect Bite

The immunologic response following insect bite tends to favor the creation of a TH2 response in that the materials known to down-regulate these responses may be used up in the initial phase of the invenomation. Toxins act primarily by the creation of excess oxidation stress.

Excess oxidation uses up the antioxidants in the local environment resulting in decreases of antioxidants such as glutathione and glutathione peroxidase. The ratio of reduced glutathione to oxidized glutathione locally affects the local immune cells. The decreased availability of reduced glutathione leads to the situation that a TH2 type of immune response becomes predominant (Peterson). Thus, the presence of insect toxin creates a situation that predisposes to chronic inflammation in the local environment.

Release of histamine is also associated with inflammation. Inflammation appears to be designed as a localized protective response which is initiated by either the injury itself such as insect toxin or the immunologic recognition and release of mediators of inflammation. These mediators serve to create constriction of the local blood vessels and release of extracellular fluids and white blood cells, which serve to dilute or wall off both the injurious agent and the injured tissue.

Inflammation is characterized by the classic signs of dolor (pain), calor (heat), rubor (swelling) and even loss of function. The word inflammation is derived from the latin words inflammation and inflammare which mean to set on fire. This description certainly describes the response felt by individuals who experience stings by insects, particularly a form of ant called the fire ant.

The fire ant, Solenopsis invicta, thought to have arrived in this country in the 1920's now ranges from Texas throughout the Southeastern portion of the United States. Fire ants can inflict multiple bites at one site and result in bites that can cause sterile psuedopustules to hypersensitivity (Ellis). Even without severe reactions, fire ant bites are associated with significant itching that can last for days.

The application of the liposomal melatonin complex of the present invention results in rapid resolution of itching and inflammation related to fire ant bite. This observation is reviewed in the case studies.

Thus histamine and melatonin are involved in stimulating the T helper cell response in what appears to be a balancing relationship. Histamine stimulates an increase in TH2 response, while melatonin down-regulates the TH2 response and increases the function of TH1 cells.

Keratinocytes form the first line of defense of the skin. The keratinocytes signal the danger of invading organisms by the production and release of cytokines and chemokines. Propionibacterium acnes (PA), along with Staphylococcus epidermidis are frequently cultured from acne lesions. However, while PA seems to contain or release mediators that stimulate pro-inflammatory responses and stimulate the chronic inflammation of acne (Bilecka). The topical application of the liposomal melatonin invention at and around sites of acne flare-up is now found in this invention to moderate the chronic inflammation related to acne.

One of the cytokines released from macrophages after contact with PA is tumor necrosis factor (TNF) (Bialecka). As TNF has been shown to sensitize cells to the effects of oxidation. Based on clinical observation of the evolution of acne lesions, the application of topical liposomal melatonin alters the usual response to the PA bacteria by favoring a path that enhances the TH1 response instead of triggering the more local tissue destructive chronic inflammatory response.

The chronic inflammatory response causes the release of cytokines such as TNF-α. The clinical theory is that TNF-α factor is an inflammatory cytokine that causes damage by generation of oxidative stress. TNF-α has been shown to sensitize cells to injury from peroxide (H₂O₂). Peroxide is an oxidant produced by various cells responding to viral infection including polymorphonuclear cells, natural killer (NK) cells and T-killer cells. The presence of TNF-α even in low concentrations increases the permeability of cells, such as endothelial cells lining the respiratory tract, to damage from H₂O₂ peroxidation. The amount of reduced glutathione contained in cells has been shown to be decreased in a concentration-dependent fashion upon exposure to TNF-α.

It appears that TNF-α decreases the availability of reduced glutathione, resulting in an increase in local oxidation stress. The formation of the oxidized form of glutathione, GSSG, can accumulate when its rate of formation exceeds the cells ability to convert it back to reduced glutathione, GSH. In this situation, GSSG can be extruded out of the cell into the extracellular space, or can form mixed disulfides with intra or extracellular proteins resulting in a net loss of total glutathione inside the affected cell (Ishii).

The resulting deficiency of glutathione leaves normal cells exposed to TNF-α induced peroxidation damage. Thus, the normal response of the immune system, in the presence of a glutathione deficiency, in fact exacerbates the symptomatic condition because the membrane of the normal cells becomes more susceptible to peroxidation damage. Peroxidation damage directed at diseased cells or infectious agents is a desired response; however, such damage directed at normal cells is undesirable.

When normal cells begin to suffer the peroxidation damage, the negative effects of TNF-α peroxidation and the reduction in cell glutathione can reinforce each other to the detriment of any cell. First, the release from the immune and epithelial cells of TNF-α is unregulated, and second, cells become progressively more sensitive to peroxidation damage as a result of continued TNF-α release, exacerbating local oxidative stress, often resulting in intensification of symptoms.

The topical application of the invention of liposomal melatonin at the site of acne eruptions will provide support to ameliorate the inflammatory activity at the site both directly and indirectly as described by melatonin's ability to up-regulate the production of antioxidants such as glutathione peroxidase and reduced glutathione. Based on clinical observation it appears that the topical application is consistent with the above explanation. In addition, a product made of the combination of melatonin and glutathione is also claimed for the treatment of acne. See examples for more details of this product.

Eczema is a disease of the skin that involves a complex immune process. Eczema and contact dermatitis are characterized by a polymorphous skin inflammation characterized at least in the acute phase by erythema, vesiculation and pruritis (SaintMezard). The mechanism remains confusing for these entities, but as they involve similar inflammatory mediators and cause similar skin changes to the examples shown, the treatment of eczema, contact dermatitis and allergic contact dermatitis are claimed for the inventions, including both the melatonin liposome complex and the combination of melatonin and glutathione in liposome complex.

Supporting the observation of the topical absorption of melatonin is a study demonstrating that the application of melatonin topically has been demonstrated to increase the level of melatonin in the circulation, although not higher than documented physiologic levels (Fischer).

Absorption of melatonin was noted to occur from either cream or aqueous solution. Melatonin's lipophilic nature was thought to explain its ability to penetrate the skin (Fischer).

Background with Respect to Acne and Skin Inflammatory Diseases

Melatonin, or N-acetyl-5-methoxytryptamine is produced primarily in the pineal gland through several biochemical steps from serotonin. Serotonin is the product of a multi-step metabolic pathway derived from L-tryptophan hydroxylation and the cofactor 6-tetrahydrobiopterin (6BH4). Hydroxytryptophan, formed from L-trytophan is decarboxylated to generate serotonin. Serotonin is best known as a neurotransmitter involved in cognition, regulation of hunger, mood, anxiety, aggression, pain, sleep and other body rhythms.

The steps involved in production of melatonin from serotonin is acetylation of serotonin to produce N-acetylserotonin, which is then methylated into melatonin by hydroxyindole-O-methyltransferase (Slominski). Melatonin is a biochemical entity that is known to be separate and distinct from serotonin in both its structure and its function. There is no suggestion made that the serotonin could be effectively applied topically for relief of itching, nor is it documented that serotonin be reliably converted to melatonin for the relief of if applied topically to a site of local inflammation and itching. The advantage of the topical liposomal melatonin is that the antioxidant and anti-inflammatory actions of melatonin are concentrated at the site of inflammatory injury to the skin cells without requiring a systemic ingestion and dissemination.

### Background for Topical Liposomal Encapsulation of Melatonin

The action of melatonin in skin lesions such as acne is multi-fold. These actions include the biochemical anti-inflammatory action described by COX-2 inhibition, of direct antioxidant action of melatonin diminishes the damage that occurs from the oxidation stress of toxins, and the modulation of immune responses. The addition of reduced glutathione to the mixture increases the immune modulation and cell protective qualities of the invention.

The delivery of the melatonin in liposomes allows the combination to penetrate the skin to the area of inflammation. Disruption of the lipid membrane by oxidation stress results in the release of active, that is unused or non-metabolized and non-oxidized melatonin at the site of the oxidation stress. The fat soluble qualities of melatonin allows it to cross tissue membrane barriers permeating local cells and within the subcellular confines of the cell (Reiter) to reach receptors and to stimulate the production of supportive antioxidant functions.

The preferred mode of the invention, a spray for topical use, allows the administration of a supraphysiologic dose at the local tissue site, which is not suggested by any prior art. The normal range of melatonin in circulation is 100 to 200 pg/mL between 2 a.m. and 5 a.m., whereas in many older people peak nocturnal levels may be only 20-40 pg/mL or even less. The blood levels of picogram amounts suggests that the levels in tissues such as the skin would be equal to or less than these levels. Thus, the topical application of liposomal melatonin in 500 microgram amounts can create the situation where there is at least a 10,000 to 100,000 amount increase of melatonin in the skin tissues. Levels such as these were required for antioxidant protection of lipid membranes (Duell).

The invention has been demonstrated to have benefit in skin inflammation and itch from insect bites and sunburn. The absorption of melatonin directly without any additive encapsulation or transformation has been demonstrated but no patent has referenced the use of melatonin for the purposes described in this application.

Keller, et al, in U.S. Pat. No. 5,891,465 propose the increased absorption of melatonin encapsulated in a liposome when delivered orally. While the data they present suggests increased absorption occurs with an oral spray of liposomal melatonin there is no reference for use on the skin. Because products supplied orally are not usually manifesting effects dermally, this product supplies the surprising benefit of liposomal contained melatonin when applied topically to the skin. The application of liposomal melatonin spray to the skin of case 1 resulted in the resolution of the itching associated with insect bite is a novel application of liposomal encapsulated melatonin as there is no reference to this usage in any of the patents or literature reviewed. No reference to this type of action appears in any of the literature or patents reviewed.

### OBJECTS OF THE INVENTION

It is an object of the invention to ameliorate the symptom of itching that occurs after insect bite, sunburn and similar irritants to the skin associated with histamine release.

It is an object of the invention to use melatonin to modify immune function to create the situation in local skin immune cells allowing a switch to a more efficient immune function such as the TH1 response following stimuli which create chronic inflammation in the skin.

It is an object of the invention to stimulate melatonin receptors in skin cells and local immune cells in skin to moderate the release of the mediators of chronic inflammation at local sites of skin inflammation.

It is an object of the invention to deliver melatonin directly to immune cells such as the macrophage. As macrophage have a predilection to ingest particulate materials (Van Rooijen) such as liposomes, so the delivery of melatonin directly to these cells, responsible for directing immune responses and participating in inflammation, is particularly effective.

It is an object of the invention to utilize the liposomal encapsulation to deliver the melatonin in an active, that is non-oxidized form, to the local skin cells and immune cells to moderate the oxidation stress effects of acute toxin exposure, the initial inflammatory response and the chronic inflammatory response to toxin and skin irritation such as sunburn.

### DETAILED DESCRIPTION OF THE PREFERRED EMBODIMENTS

The preferred mode of the invention is the liposomal encapsulation of melatonin as this allows melatonin absorption and keeps the melatonin stable inside the liposome. The liposome protects the melatonin from exposure to oxygen from air or from an aqueous solution that would promote oxidation of the melatonin.

The preferred mode of this invention is the encapsulation of melatonin into liposomes. The advantage of the encapsulation method includes improved penetration into skin facilitated by the liposome, and stabilization of melatonin in the reduced state, while allowing suspension in an aqueous state.

This invention has the advantage of ease of application in either spray or cream mixtures.

While the absorption of melatonin could lead to a sleepiness side effect, no sleepiness was noted in the case subjects. This is probably due to either the local metabolism of melatonin or to the metabolism of melatonin in the liver, which is its primary site of metabolism (Slominski).

Synthetic melatonin is used in the preferred embodiment, but natural, animal derived melatonin may also be used. References to melatonin in this application refer to both the synthetic and natural forms of melatonin.

The formation of the invention may include melatonin or metabolites of melatonin. The most preferred compound is melatonin itself. Other preferred compounds, which can be obtained in synthetic processes, for use in the compositions of the invention are 5-methoxytryptamine, 5-methoxytryptophan, 5-methoxytryptophol, 5-methoxyindole-3-acetic acid and 6-hydroxy-melatonin. Also included are the active metabolites of melatonin N1-acetyl-N2-formyl-5-methoxykynuramine (AFMK) and N1-acetyl-5-methoxykynuramine (AMK). The term “melatonin” is used to designate both the actual melatonin and the chemical homologues or derivatives thereof.

### Example 1

### Liposomal Melatonin Spray 500 Micrograms Per Spray

Melatonin liposome combination designed to yield a spray with an individual volume of 0.65 cc, yielding approximately 500 mcg. melatonin per spray.

| Component | % w/w |
|---|---|
| Deionized Water | 75.075 |
| Glycerin | 15.00 |
| Lecithin | 1.50 |
| Potassium Sorbate | 0.10 |
| (optional spoilage | |
| retardant) | |
| melatonin | 0.075 |

A lipid mixture having components lecithin, and glycerin were commingled in a large volume flask and set aside for compounding.

In a separate beaker, a water mixture having water, glycerin, melatonin were mixed and heated to 50.degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

### Example 2

### Liposomes Containing a Combination of Melatonin and Reduced Glutathione

Melatonin liposome combination designed to yield a spray with an individual volume of 0.65 cc, yielding approximately 500 mcg. melatonin per spray and approximately 54 mg of glutathione.

| Component | % w/w |
|---|---|
| Deionized Water | 75.075 |
| Glycerin | 15.00 |
| Lecithin | 1.50 |
| Potassium Sorbate | 0.10 |
| (optional spoilage | |
| retardant) | |
| melatonin | 0.075 |
| Glutathione (reduced) | 8.25 |

A lipid mixture having components lecithin, and glycerin were commingled in a large volume flask and set aside for compounding.

In a separate beaker, a water mixture having water, glycerin, glutathione and melatonin were mixed and heated to 50 degree. C.

The water mixture was added to the lipid mixture while vigorously mixing with a high speed, high shear homogenizing mixer at 750-1500 rpm for 30 minutes.

The homogenizer was stopped and the solution was placed on a magnetic stirring plate, covered with parafilm and mixed with a magnetic stir bar until cooled to room temperature. Normally, a spoilage retardant such as potassium sorbate or BHT would be added. The solution would be placed in appropriate dispenser for ingestion as a liquid or administration as a spray.

Analysis of the preparation under an optical light microscope with polarized light at 400× magnification confirmed presence of both multilamellar lipid vesicles (MLV) and unilamellar lipid vesicles.

The preferred embodiment includes the variations of the amount of glutathione to create less concentrated amounts of glutathione. The methods of manufacture of oral liposomal encapsulation of melatonin, with for the purpose of this invention the inclusion of glutathione, using the method described in Keller et al, U.S. Pat. No. 5,891,465 are incorporated into this description.

### Example 3

### Liposomes Containing Melatonin in a Combination Designed for More Efficient Topical Preparation

Melatonin in a glycerol distearate (GSD) liposome designed to yield a spray with an individual volume of 0.65 cc, yielding approximately 150 mcg. melatonin per spray and approximately.

| Component | % w/w |
|---|---|
| Deionized Water | 77.14 |
| Glycerin | 4.00 |
| melatonin | 0.023 |
| Pyridixine HCl | 0.1 |
| Isopropyl Palmitate | 5.0 |
| Propylene Glycol | 4 |
| Transcutel CG | 8 |
| Polysorbate 60 | 0.5 |
| GDS-12 | 1.0 |

GSD-12 denotes GSD refers to glycerol distearate (1,2-distearoyl-rac-glycerol-3-dodecaethylene glycol), which is described in Keller et al U.S. Pat. No. 6,958,160, published Oct. 25, 2005, and which is incorporated in this description in its entirety.

A mixture having components purified water, melatonin, glycerin and pyridoxine HCL are commingled in a large volume flask and heated to 60° C. Mix until solids dissolved and the mixture is uniform.

In a separate beaker, a mixture containing isopropyl palmitate, propelyne glycol, Transcutol CG, Polycorbate 60 and GDS-12 are heated to 60° C. and mixed until uniform.

Combine the two mixtures and mix until uniform.

At 40° add Uniphen and titrate with triethanolamine (TEA) to a pH of 5.50.

Examination by optical microscope at 100 times and 600 times shows a suspension of multilamellar liposomes.

An embodiment includes the variations of the amount of glutathione to create less concentrated amounts of glutathione. The methods of manufacture of oral liposomal encapsulation of melatonin, with for the purpose of this invention the inclusion of glutathione, using the method described in Keller et al, U.S. Pat. No. 5,891,465 are incorporated into this description.

The preferred embodiment includes the variations of the amount of GSD-12 up to 10%. At the 10% concentration the combination will be in the form of a cream and will be applied as a cream to the affected area. The method described by Keller et al in U.S. Pat. No. 6,958,160, published Oct. 25, 2005, are incorporated in this description. A typical pharmaceutical carrier, namely propylene glycol can be added to this embodiment to act as an adhesive to the skin. This combination allows for increased contact of the preparation with the skin and a lower amount of melatonin to be effective. Other known pharmaceutical carriers with adhesion characteristics known to person of ordinary skill in the art published in lead texts may be used.

### General Dosing

The preferred application schedule of the invention for the treatment of itching on the skin secondary to an insect bite is a single spray of either the 500 micrograms (mcg) of liposomal melatonin or the 150 mcg of the adhesive liposomal melatonin spray.

The application amount sprayed should be sufficient to cover the affected area. An application of is to be repeated every one to hours until symptoms are relieved. Once symptom relief is achieved, the dose is repeated immediately upon the return of symptoms. The anticipated amount to be taken is 4 to 5 sprays in 24 hours. If a single spray is not sufficient to cover the affected area, additional sprays to cover the area are recommended, as set forth in the case examples provided. For symptoms of itching, irritation and pain due to sunburn a sufficient number of single dose sprays to cover the affected area is recommended. The application may be repeated in 1 to 4 hours as needed to relieve the symptoms. The applications should be continued until the symptoms have of sunburn have been resolved. It is anticipated that this may require 4 to 6 applications in a 24 hour period.

If symptoms recur in the following 24 hours the regimen may be repeated as stated.

One spray of the liposomal melatonin invention contains 500 micrograms of melatonin, while one spray of the adhesive liposomal melatonin spray invention contains 150 micrograms of melatonin

### Dosing Schedule for the Treatment of Sunburn

The initial application will be determined by the area of the body affected by excessive sun exposure. The initial application should use the minimum sprays needed to cover the area affected using repeated applications of single sprays. The number of sprays will be determined by the area affected.

The amount and frequency of doses may be decreased as the individual begins to improve. The period of treatment is usually 24 to 48 hours.

Application of the liposomal preparation of melatonin results in a rapid reduction of the symptoms of itching as related in the examples cited. The mechanism may be related to one or more of the methods described. The application of the invention to the skin of an affected area results in the addition of melatonin to the local skin area and has a number of avenues to facilitate the restoration of normal skin cell and immune cell function that results in the reduction of symptoms related to insect bite, sunburn and other irritant sources of histamine release.

The dosing schedule for the preparation containing liposomal encapsulation of melatonin and glutathione, labeled preparation 2 and 3 are the same as the dosing schedules recommended for the applications of preparation 1.

### Dosing for the Treatment of Acne

The combination of melatonin and glutathione in liposomes is applied by spray or cream to the site of acne inflammation morning, afternoon and before bed.

The amount of each spray is 500 mcg. of melatonin encapsulated in the liposome or 150 mcg of melatonin in the adhesive liposomal melatonin spray.

Initial application should be done immediately upon recognition that an acne eruption is occurring and should continue with the three times a day application until the lesion has resolved.

The action of melatonin in skin lesions such as acne is multi-fold. These actions include the biochemical anti-inflammatory action described by COX-2 inhibition, of direct antioxidant action of melatonin diminishes the damage that occurs from the oxidation stress of toxins, and the modulation of immune responses. The addition of reduced glutathione to the mixture increases the immune modulation and cell protective qualities of the invention.

### Topical Liposomal Melatonin

### CASE EXAMPLES

Some examples of trials of topical liposomal melatonin follow.

### Topical Liposomal Melatonin in the Management of Skin Itching and Erythema After Insect Bite

Case 1.

GG, a 58 year old woman with insect (possibly spider) bite on left flank. Itching and irritation present around 4 small raised popular lesions on skin. Individual has very fair skin and history of bites and irritants causing prolonged irritation to skin.

Liposomal melatonin sprayed onto skin. 2 sprays over area and the resulting liquid spread gently over skin.

GG reports that about 15 minutes after the spray she noticed that the affected area was no longer itching. The effect lasted at least 6 hours. Another application was applied. No drowsiness was observed.

No further application for 18 hours when a small amount of itching was noted. These symptoms abated after another application of liposomal melatonin.

The individual noted approximately 2 weeks later that the sites of the bites were healing with less redness and resolving more quickly than bites of this type generally do.

Case 2.

TG, A 50 year old man experienced fire ant bites approximately 36 hours prior to applying the invention. He had approximately 6 raised, red papules on the inside of the right ankle that were accompanied by continued itching. No other treatments had been applied.

The invention was applied by 3 sprays over the affected area.

Approximately 10 minutes later TG noted that he no longer felt the itching. He continued the applications every 4 to 6 hours over the next 48 hours and noted that there was no return of the itching.

Case 3

TG1, a 28 year old woman was bitten by numerous fire ants around the lower portion of the leg on both sides. The bites were in the early inflammatory phase, associated with pain, about 15 minutes after the bites when she was offered the present invention. Within 15 to 20 minutes of the application of the liposomal melatonin she noted 90% resolution of the pain and irritation from bites. The improvement lasted for over 2 hours, when symptoms began to recur.

Case 4

JG, a 35 year old man with a long history of sensitivity to poison oak development redness and itching over the right eye lid. The symptoms developed following contact with a dog that had run through an area containing poison oak several hours before and was typical of the onset of the symptoms that he previously experienced.

The liposomal melatonin invention was applied in 2 sprays over the right eye area. After 3 minutes the itching was improved, but still present. A second application was sprayed over the right eye area several minutes later consisting of two sprays. Care was taken to avoid application directly to the mucosa of the eye.

After 15 minutes the affected area of the right eye was no longer itching. After 30 minutes symptoms of the irritation were barely noticeable.

Eight hours later JG had mild irritation and mile edema of the right eyelid. There was minimal to no itching present. The left eye, which had mild symptoms and no treatment had moderate (more swelling than the left eyelid) and mild to moderate itching.

Case 5

Two female children ages 7 and 10 experience regulation interaction mosquito bites and fire ant stings at several sites on their legs. The embodiment of the invention described as adhesive liposomal spray is applied to new mosquito bites on several occasions and on each occasion the itching from the mosquito bite is reduced promptly. A protocol for management of the itching from previous bites involves the application of the spray in the morning to each of several sites of residual irritation due to the mosquito bite or previous fire ant bite. It is observed that this ameliorates the itching for approximately 8 hours. A second application is applied to the affected areas which again gives several hours of relief. No drowsiness is noted from this protocol.

Topical Liposomal Melatonin in the Management of Skin Itching and Pain after Sunburn

Case 6.

HG. a 58 year old woman with a history of sunburn approximately 72 hours prior to evaluation. The sunburn occurred over the area of the upper and inner thighs after sitting in a small boat on a somewhat overcast day without applying sun screen. The individual noted that the sunburn had been very painful in the first 48 hours. She estimated the initial pain as approximately a 10/10 rating initially. At the time of evaluation she rated the pain as 4/10 and accompanied by itching at moderate and irritation at moderate.

The right thigh had desquamating skin, was red and erythematous and had more prominent itching and pain than the left thigh. The invention, topical liposomal melatonin spray, was applied to the right thigh burn area.

Ten minutes later HG noted that the pain had reduced to 0/10 on the right thigh, while it continued on the left thigh. The itching had reduced to negligible from moderate and the irritation as minimal. She noted that there was significant perception of improvement on the right side, where the invention was applied compared to the left, untreated side.

HG noted continued improvement in the symptoms described and continued to apply the topical liposomal melatonin spray invention every one to two hours to selected sites of itching and pain over the next 24 to 48 hours and the symptoms resolved.

The embodiments represented herein are only a few of the many embodiments and modifications that a practitioner reasonably skilled in the art could make or use. The invention is not limited to these embodiments. Alternative embodiments and modifications which would still be encompassed by the invention may be made by those skilled in the art, particularly in light of the foregoing teachings. Therefore, the following embodiments and claims are intended to cover any alternative embodiments, modifications or equivalents which may be included within the spirit and scope of the invention as described and claimed.

### Topical Liposomal Melatonin in the Management of Skin Redness, Itching, Pain and Irritation Related to Radiation Therapy

Case 7

MR, a 58 year old woman undergoing radiation therapy for breast cancer reports redness and local irritation on the lateral surface of the right breast after undergoing 15 radiation treatments. MR began using the liposomal spray melatonin, 500 mcg per spray, using 3 sprays to cover the affected area of redness. Within two days of beginning the spray she noted decreased redness and decreased irritation of the skin at the site of the radiation. She was able to complete an additional 15 radiation treatments without discomfort and minimal skin redness and irritation.

### REFERENCES

1. Allegra M, Reiter R J, Tan D X, Gentile C, Tesoriere L, Livrea M A. The chemistry of melatonin's interaction with reactive species. J Pineal Res. 2003 January; 34(1):1-10. PMID:
2. Bialecka A, Mak M, Biedron R, Bobek M, Kasprowicz A, Marcinkiewicz J. Different pro-inflammatory and immunogenic potentials of Propionibacterium acnes and Staphylococcus epidermidis: implications for chronic inflammatory acne. Archivum immunologiae et therapiae experimentalis. 2005 January-February; 53(1):79-85. PMID: 15761379
3. Duell P B, Wheaton D L, Shultz A, Nguyen H. Inhibition of LDL oxidation by melatonin requires supraphysiologic concentrations. Clin Chem. 1998 September; 44(9):1931-6. PMID: 9732979
4. Glover R A, Bailey C S, Barrett K E, Wasserman S I, Gigli I. Histamine release from rodent and human mast cells induced by protoporphyrin and ultraviolet light: studies of the mechanism of mast-cell activation in erythropoietic protoporphyria. Br J Dermatol. 1990 April; 122(4):501-12. PMID: 2337518
5. El-Shenawy S M, Abdel-Salam O M, Baiuomy A R, El-Batran S, Arbid M S. Studies on the anti-inflammatory and anti-nociceptive effects of melatonin in the rat. Pharmacol Res. 2002 September; 46(3):235-43. PMID: 12220966
6. Ellis A K, Day J H. Clinical reactivity to insect stings. Current Opinion Allergy Clinical Immunology. 2005 August; 5(4):349-354 PMID: 15985818
7. Fischer T W, Greif C, Fluhr J W, Wigger-Alberti W, Elsner P. Percutaneous penetration of topically applied melatonin in a cream and an alcoholic solution. Skin Pharmacol Physiol. 2004 July-August; 17(4):190-4 PMID: 15258450
8. Gurlek A, Aydogan H, Parlakpinar H, Bay-Karabulut A, Celik M, Sezgin N, Acet A. Protective effect of melatonin on random pattern skin flap necrosis in pinealectomized rat. J Pineal Res. 2004 January; 36(1):58-63. PMID: 14675131
9. Handwerker H O, Forster C, Kirchhoff C. Discharge patterns of human C-fibers induced by itching and burning stimuli. Journal of Neurophysiol. 1991 July; 66(1):307-15 PMID: 1919673
10. Ishii Y, Partridge C A, Del Vecchio P J, Malik A B. Tumor necrosis factor-alpha-mediated decrease in glutathione increases the sensitivity of pulmonary vascular endothelial cells to H2O2. Journal Clinical Investigation 1992 March; 89(3):794-802. PMID: 1541673
11. Kam P C, Tan K H. Pruritus—itching for a cause and relief? Anaesthesia. 1996 Dec.(12):1133-8. PMID: 9038449
12. Kiefer W, Dannhardt G. COX-2 inhibition and the control of pain. Current Opinion and Investigational Drugs 2002; 2: 1348-1358. PMID: 12498012
13. Mayo J C, Sainz R M, Tan D X, Hardeland R, Leon J, Rodriguez C, Reiter R J. Anti-inflammatory actions of melatonin and its metabolites, N1-acetyl-N2-formyl-5-methoxykynuramine (AFMK) and N1-acetyl-5-methoxykynuramine (AMK), in macrophages. Journal Neuroimmunology. 2005 August; 165(1-2):139-49. PMID: 15975667
14. Packard K A, Khan M M. Effects of histamine on TH1/TH2 cytokine balance. International Immunopharmacology. 2003 July; 3(7):909-20. PMID: 12810348
15. Peterson J D, Herzenberg L A, Vasquez K, Waltenbaugh C. Glutathione levels in antigen-presenting cells modulate TH1 versus TH2 response patterns. Proceedings National Academy Sciences USA. 1998 Mar. 17; 95(6):3071-6. PMID: 9501217
16. Maestroni G J The immunotherapeutic potential of melatonin. Expert opinion on investigational drugs. 2001 March; 10(3):467-76 PMID: 11227046
17. Simone D A, Alreja M, LaMotte R H Psychophysical studies of the itch sensation and itchy skin (“alloknesis”) produced by intracutaneous injection of histamine. Somatosensory & Motor Research 1991; 8(3):271-9 PMID: 1767623
18. Reiter R J, Tan D X, Leon J, Kilic U, Kilic E. When melatonin gets on your nerves: its beneficial actions in experimental models of stroke. Experimental Biology and Medicine (Maywood). 2005 February; 230(2):104-17. Review. PMID: 15673559
19. Rothman S. Physiology of itching. Physiological Reviews 1941; 21:357-81. http://physrev.physiology.org/cgi/reprint/21/2/357?ijkey=dc3a779766f32f4d92500cl bf9 79e06b3dff94bc&keytype2=tf_ipsecsha
20. Saint-Mezard P, Rosieres A, Krasteva M, Berard F, Dubois B, Kaiserlian D, Nicolas J F. Allergic contact dermatitis. Eurj Dermatol. 2004 September-October; 14(5):284-95. Review. PMID: 15358566
21. Schmelz M, Schmidt R, Bickel A, Handwerker H O, Torebjork H E. Specific C-receptors for itch in human skin. Journal Neuroscience. 1997 October 15; 17(20):8003-8 PMID:
22. Slominski A, Wortsman J, Desmond J. The cutaneous serotoninergic/melatoninergic system: securing a place under the sun. Federation of American Societies for Experimental Biology, Journal. 2005 February; 19(2):176-94 PMID:
23. Twycross R, Greaves M W, Handwerker H, Jones E A, Libretto S E, Szepietowski J C, Zylicz Z. Itch: scratching more than the surface. Quarterly Journal of Medicine. 2003 January; 96(1):7-26 PMID: 12509645
24. Van Rooijen N, The liposome-mediated macrophage ‘suicide’ technique. Journal Immunological Methods. 1989 Nov. 13; 124(1):1-6. PMID: 2530286

What is claimed is:

1. A pharmaceutical composition enabling delivery to the skin after topical application of a therapeutically effective amount of melatonin comprising:
a therapeutic dose of melatonin stabilized in a liposome pharmaceutical carrier capable of being delivered to the skin as a spray at the site of an insect envenomation such as a mosquito bite or fire ant bite in sufficient quantity to relieve symptoms of itch and irritation associated with the physiologic response to the envenomation.

2. The pharmaceutical composition according to claim 1, further comprising:
liposomes derived from lecithin.

3. The pharmaceutical composition according to claim 1, further comprising:
liposomes derived from using glycerol distearate (1,2-distearoyl-rac-glycerol-3-dodecaethylene glycol).

4. A pharmaceutical composition enabling delivery to the skin after topical application of a therapeutically effective amount of melatonin comprising:
a therapeutic dose of melatonin stabilized in a liposome pharmaceutical carrier having liposomes derived using glycerol distearate (1,2-distearoyl-rac-glycerol-3-dodecaethylene glycol) capable of being delivered to the skin at the site of an insect envenomation such as a mosquito bite or fire ant bite in sufficient quantity to relieve symptoms of itch and irritation associated with the physiologic response to the envenomation.

5. The composition according to claim 4, further comprising:
a pharmaceutical carrier with skin-adhesion characteristics.

6. The composition according to claim 5, further comprising:
said pharmaceutical carrier with skin-adhesion characteristics being polypropylene glycol.

7. A pharmaceutical composition for treatment of inflammatory skin conditions arising from immunologic response, comprising:
a therapeutic dose of melatonin stabilized in a liposome pharmaceutical carrier treatment of inflammatory skin conditions capable of being delivered to the skin in sufficient quantity to relieve skin conditions arising from immunologic response.

8. The pharmaceutical composition according to claim 7, further comprising:
liposomes derived from lecithin.

9. The composition according to claim 8, further comprising:
a pharmaceutical carrier with skin-adhesion characteristics.

10. The composition according to claim 9, further comprising:
said pharmaceutical carrier with skin-adhesion characteristics being polypropylene glycol.

11. The pharmaceutical composition according to claim 7, further comprising:
liposomes derived from using glycerol distearate (1,2-distearoyl-rac-glycerol-3-dodecaethylene glycol).

12. The composition according to claim 11, further comprising:
a pharmaceutical carrier with skin-adhesion characteristics.

13. The composition according to claim 12, further comprising:
said pharmaceutical carrier with skin-adhesion characteristics being polypropylene glycol.

14. The composition according to claim 7, further comprising:
said immunologic reaction being psoriasis.

15. The composition according to claim 7, further comprising:
said immunologic reaction being eczema.

16. A pharmaceutical composition for treatment of inflammatory skin conditions arising from immunologic response, comprising:
a therapeutic dose of melatonin stabilized in a liposome pharmaceutical carrier for treatment of inflammatory skin conditions having liposomes derived using glycerol distearate (1,2-distearoyl-rac-glycerol-3-dodecaethylene glycol) capable of being delivered to the skin in sufficient quantity to relieve skin conditions arising from immunologic response.

17. The composition according to claim 16, further comprising:
a pharmaceutical carrier with skin-adhesion characteristics.

18. The composition according to claim 17, further comprising:
said pharmaceutical carrier with skin-adhesion characteristics being polypropylene glycol.

19. The composition according to claim 16, further comprising:
said immunologic reaction being psoriasis.

20. The composition according to claim 16, further comprising:
said immunologic reaction being eczema.

21. A pharmaceutical composition for treatment of an acne eruption, comprising:
a therapeutic dose of melatonin stabilized in a liposome pharmaceutical carrier treatment of inflammatory skin conditions having liposomes derived using glycerol distearate (1,2-distearoyl-rac-glycerol-3-dodecaethylene glycol) capable of being delivered to the skin in sufficient quantity to relieve symptoms of said acne eruption.

22. The composition according to claim 21, further comprising:
a pharmaceutical carrier with skin-adhesion characteristics.

23. The composition according to claim 22, further comprising:
said pharmaceutical carrier with skin-adhesion characteristics being polypropylene glycol.

24. A pharmaceutical composition for treatment of radiation treatment induced dermatitis comprising:
a therapeutic dose of melatonin stabilized in a liposome pharmaceutical carrier capable of being delivered to the skin as a spray.

25. A pharmaceutical composition for treatment of radiation treatment induced dermatitis comprising:
a therapeutic dose of melatonin stabilized in a liposome pharmaceutical carrier having liposomes derived using glycerol distearate (1,2-distearoyl-rac-glycerol-3-dodecaethylene glycol) capable of being delivered to the skin in sufficient quantity to relieve said dermatitis.

26. The composition according to claim 25, further comprising:
a pharmaceutical carrier with skin-adhesion characteristics.

27. The composition according to claim 26, further comprising:
said pharmaceutical carrier with skin-adhesion characteristics being polypropylene glycol.

28. A pharmaceutical composition for treatment of sunburn, comprising:
a therapeutic dose of melatonin stabilized in a liposome pharmaceutical carrier having liposomes derived using glycerol distearate (1,2-distearoyl-rac-glycerol-3-dodecaethylene glycol) capable of being delivered to the skin in sufficient quantity to relieve said sunburn.

29. The composition according to claim 28, further comprising:
a pharmaceutical carrier with skin-adhesion characteristics.

30. The composition according to claim 29, further comprising:
said pharmaceutical carrier with skin-adhesion characteristics being polypropylene glycol.

31. A composition for relieving dermatitis, including from sunburn, comprising:
a therapeutic dose of melatonin stabilized in a liposome pharmaceutical carrier; and
a therapeutic dose of reduced glutathione stabilized in a liposome pharmaceutical carrier;
said therapeutic doses being encapsulated in liposomes derived from lecithin capable of being delivered to the skin in sufficient quantity to relieve said dermatitis.

32. The composition according to claim 31, further comprising:
a pharmaceutical carrier with skin-adhesion characteristics.

33. The composition according to claim 32, further comprising:
said pharmaceutical carrier with skin-adhesion characteristics being polypropylene glycol.

34. A composition for relieving dermatitis, including from sunburn, comprising:
a therapeutic dose of melatonin stabilized in a liposome pharmaceutical carrier; and
a therapeutic dose of reduced glutathione stabilized in a liposome pharmaceutical carrier;
said therapeutic doses being encapsulated in liposomes derived using glycerol distearate (1,2-distearoyl-rac-glycerol-3-dodecaethylene glycol) capable of being delivered to the skin in sufficient quantity to relieve said dermatitis.

35. The composition according to claim 34, further comprising:
a pharmaceutical carrier with skin-adhesion characteristics.

36. The composition according to claim 35, further comprising:
said pharmaceutical carrier with skin-adhesion characteristics being polypropylene glycol.
